(19) United States
(12) Patent Application Publication
(10) Pub. No.: US 2024/0335237 A1
(43) Pub. Date: Oct. 10, 2024

(54) System and Method for Mapping an Anatomical Pathway

(71) Applicant: Bard Access Systems, Inc., Salt Lake City, UT (US)

(72) Inventors: Steffan Sowards, Salt Lake City, UT (US); William Robert McLaughlin, Bountiful, UT (US)

(21) Appl. No.: 18/132,891

(22) Filed: Apr. 10, 2023

(51) Int. Cl. A61B 34/20; A61B 5/06; G16H 10/60; G16H 40/63

(52) U.S. Cl. CPC A61B 34/20; A61B 5/062; G16H 10/60; G16H 40/63; A61B 2034/2061

(57) ABSTRACT

A medical network system is communicatively coupled with a number medical device tracking systems that utilize magnetic systems to track elongate medical devices along anatomical pathways within patients having differing physical attributes, such as height and weight. The medical network system collects tracking records from the medical device tracking systems and applies machine learning and/or artificial intelligence technics to the tracking record data to correlate recorded anatomical pathways with the differing physical attributes. The medical network system receives a patient value set of physical attributes for a specific patient from a medical device tracking system and provides a predicted anatomical pathway based on the correlation and the patient value set. The medical device tracking systems tracts the elongate medical device with respect to the predicted anatomical pathway during placement. The medical device tracking systems may also utilize fiber optic shape sensing systems to track the placement of elongate medical.

### BACKGROUND

The placement of the vascular devices has recently benefited from magnetic tracking systems that track the position of the vascular during advancement along the vasculature. The tracking system have enabled the clinician to visually monitor the position of the vascular device during the placement so that the clinician may detect a condition of incorrect placement of the device. A magnetic sensing unit placed on the patient can provide some anatomical reference for the position of the device. However, due to the significant physiologic differences between patients, the anatomical reference provided by the magnetic sensing unit can allow for undetected misplacement of the device with respect to a desired anatomical pathway. Such misplacement can result is patient harm and increased medical expense.

Disclosed herein are systems and methods of displaying a predicted anatomical pathway for the device to provide the clinician with a greater assurance that the device is advanced along the desired anatomical pathway.

### SUMMARY

Disclosed herein is a medical system that, according to some embodiments, includes (i) an elongate medical device configured for advancement along an anatomical pathway within a patient and (ii) a tracking system configured to track the elongate medical device within the patient, where the tracking system includes a console. The console includes a processor and a memory having logic stored thereon that, when executed by the processor performs operations of the medical system that include tracking a position the elongate medical device with respect to a predicted anatomical pathway stored in the memory.

In some embodiments, the anatomical pathway includes a vascular pathway. In some embodiments, the vascular pathway includes at least one of a lower one third of a superior vena cava or Cavo-atrial junction, and in some embodiments, the vascular pathway includes an azygos vein.

In some embodiments, the predicted anatomical pathway is defined based on a number of patient attributes.

In some embodiments, the operations further include providing a notification when the position the elongate medical device deviates from the predicted anatomical pathway.

In some embodiments, the operations further include transmitting the patient attributes to a network module and receiving the predicted anatomical pathway from the network module.

In some embodiments, the operations further include making a record of the position of the elongate medical device after advancement along the anatomical pathway and transmitting the record to the network module.

In some embodiments, the tracking system includes a magnetic tracking system that includes a number of magnetic elements disposed along the elongate medical device, where the magnetic elements define a number of magnetic fields. The magnetic tracking system further includes a number of magnetometers placed on the patient and coupled with console, where the magnetometers are configured to detect the magnetic fields. In such embodiments, tracking the position the elongate medical device includes determining positions of the magnetic elements in three-dimensional space with respect to the magnetometers.

In some embodiments, the tracking system includes a fiber optic shape sensing system that includes an optical fiber extending along the elongate medical device. The optical fiber is operatively coupled with the console and the optical fiber includes a number of optical fiber cores, where each optical fiber core includes a plurality of fiber Bragg gratings configured to determine a strain in the optical fiber. In such embodiments, tracking the position the elongate medical device further includes determining a three-dimensional shape of the elongate medical device based on the strain.

Also disclosed herein is a medical network system that, according to some embodiments, includes a number of medical device tracking systems communicatively coupled with a network system module. The network system module includes a console including a processor and a memory having logic stored thereon that, when executed by the processor performs operations that include receiving a plurality of tracking records from the medical device tracking systems. Each tracking record includes (i) a patient value set having a value for each of a number of physical attributes of a patient and (ii) a position of an elongate medical device disposed along a defined anatomical pathway within the patient, where the position of the elongate medical device is linked with the patient value set. The operations further include processing the plurality of tracking records to define a correlation between the positions of the elongate medical device and the patient value sets.

In some embodiments of the network system, each patient value set includes a number of physical of attributes of the patients, where the physical of attributes include height, weight, sex, age, body mass index, race, or any combination thereof.

In some embodiments of the network system, the operations further include receiving a new patient value set from one of the medical device tracking systems and transmitting a predicted anatomical pathway to the one of the medical device tracking systems, where the predicted anatomical pathway is based on the correlation between the positions of the elongate medical device and the patient value sets and the new patient value set.

In some embodiments of the network system, the predicted anatomical pathway includes a confidence level, and the operations further include receiving additional tracking records from the medical device tracking systems and processing the additional tracking records to further define the correlation between the positions of the elongate medical device and the patient value sets, thereby increasing the confidence level.

In some embodiments of the network system, at least one of the medical device tracking systems includes a magnetic tracking system that includes a number of magnetic elements disposed along the elongate medical device, where the magnetic elements define a number of magnetic fields; and a number of magnetometers placed on the patient and coupled with console, where the magnetometers are configured to detect the magnetic fields. In such embodiments, detecting the magnetic fields determines the positions of the magnetic elements in three-dimensional space with respect to the magnetometers, thereby determining the position of the elongate medical device within the patient.

In some embodiments, at least one of the medical device tracking systems includes a fiber optic shape sensing system, that includes an optical fiber extending along the elongate medical device. The optical fiber is operatively coupled with the console, and the optical fiber includes a number of optical fiber cores, where each optical fiber core includes a plurality of fiber Bragg gratings configured to determine a strain of the optical fiber. The strain of the optical fiber determines a three-dimensional shape of the elongate medical device within the patient, and the tracking record includes the shape of the elongate medical device, and processing the plurality of tracking records to define a correlation between the positions of the elongate medical device and the patient value sets includes defining a correlation between the shapes of the elongate medical device and the patient value sets.

Also disclosed herein is a system method of mapping an anatomical pathway for an elongate medical device that, according to some embodiments, includes collecting a plurality of tracking records from a number of medical device tracking systems. Each tracking record includes a patient value set having a value for each of a number of physical attributes of a patient and a position of an elongate medical device disposed along a defined anatomical pathway within the patient, where the position of the elongate medical device is linked with the patient value set. The system method further includes (i) processing the plurality of tracking records to define a correlation between the positions of the elongate medical device and the patient value sets, (ii) receiving a new patient value set from one of the medical device tracking systems, and (iii) defining a predicted anatomical pathway based on the correlation and the new patient value set.

In some embodiments, the system method further includes (i) collecting additional tracking records from the medical device tracking systems, (ii) processing the additional tracking records, and (iii) updating the correlation in accordance with the additional tracking records.

In some embodiments of the system method, the medical device tracking systems include a magnetic tracking system, including a number of magnetic elements disposed along the elongate medical device, where the magnetic elements define a number of magnetic fields; and a number of magnetometers placed on the patient, where the magnetometers are configured to detect the magnetic fields. In such embodiments, detecting the magnetic fields determines the positions of the magnetic elements in three-dimensional space with respect to the magnetometers, thereby determining the position of the elongate medical device within the patient.

In some embodiments of the system method, the medical device tracking system includes a fiber optic shape sensing system that includes an optical fiber extending along the elongate medical device. The optical fiber includes a number of optical fiber cores, where each optical fiber core includes a plurality of fiber Bragg gratings configured to determine a strain of the optical fiber, and further where the strain of the optical fiber determines a three-dimensional shape of the elongate medical device within the patient. In such embodiments, the tracking record includes the shape of the elongate medical device, and processing the plurality of tracking records to define a correlation between the positions of the elongate medical device and the patient value sets includes defining a correlation between the shapes of the elongate medical device and the patient value sets.

These and other features of the concepts provided herein will become more apparent to those of skill in the art in view of the accompanying drawings and following description, which describe particular embodiments of such concepts in greater detail.

### DRAWINGS

FIG. 1 illustrates a medical device tracking system, in accordance with some embodiments.

FIG. 2 illustrates the medical device tracking system of FIG. 1 in use with a patient, in accordance with some embodiments.

FIG. 3 illustrates the elongate medical device of FIG. 1 advanced along an anatomical pathway of the patient, in accordance with some embodiments.

FIG. 4A illustrates a medical network system, in accordance with some embodiments.

FIG. 4B illustrates a database table of the medical network system of FIG. 4A, in accordance with some embodiments.

FIG. 5 illustrates a screen shot of the medical device tracking system of FIG. 1 showing a predicted anatomical pathway defined by the medical network system of FIG. 4A, in accordance with some embodiments.

FIG. 6 illustrates a block diagram of a system method of the medical network system of FIG. 4A, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The phrases “connected to,” “coupled with,” and “in communication with” refer to any form of interaction between two or more entities, including but not limited to mechanical, electrical, magnetic, electromagnetic, fluid, and thermal interaction. Two components may be coupled with each other even though they are not in direct contact with each other. For example, two components may be coupled with each other through an intermediate component.

The terms “proximal” and “distal” refer to opposite ends of a medical device, including the devices disclosed herein. As used herein, the proximal portion of a medical device is the portion nearest a practitioner during use, while the distal portion is the portion at the opposite end. For example, the distal end of an elongate medical device is defined as the end closest to the patient during utilization of the elongate medical device.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

Unless defined otherwise, all technical and scientific terms used herein have the same meaning as commonly understood by those of ordinary skill in the art. References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

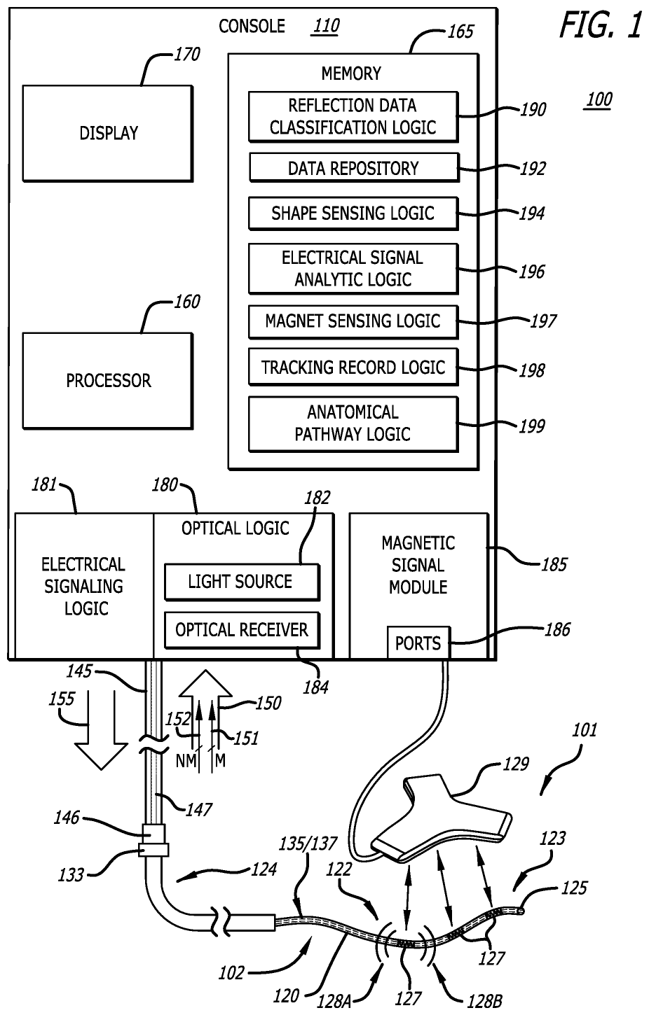

FIG. 1 is an illustrative embodiment of a medical device tracking system 100 that generally includes (i) an elongate medical device configured for advancement along an anatomical pathway within a patient and (ii) a system for tracking the elongate medical device (or more specifically, tracking the position of the elongate medical device) along the anatomical pathway. The medical device tracking system 100 includes one or both of a magnetic location system (MLLS) 101 and the shape sensing system 102, where the shape sensing system 102 is configured to determine a shape of the elongate medical device.

As shown, the system 100 generally includes a console 110, an elongate medical device 120 operatively coupled with the console 110 and a magnetic field sensor 129 also coupled with the console 110. The elongate medical device 120 may include a guidewire, a stylet, a catheter or any medical device suitable for advancement along an anatomical pathway within patient, such as a vasculature, for example. The elongate medical device 120 extends between a distal tip 123 and a console connector 133 at a proximal end 124. The console connector 133 enables the elongate medical device 120 to be operably connected to the console 110 via an interconnect 145 including one or more optical fibers 147 (hereinafter, “optical fiber(s)”). In some embodiments, an optional conductive medium of the elongate medical device 120 may be terminated by a single optical/electric connector 146 (or terminated by dual connectors). Herein, the connector 146 is configured to engage (mate) with the console connector 133 to allow for the propagation of light between the console 110 and the elongate medical device 120.

An exemplary implementation of the console 110 includes a processor 160, a memory 165, a display 170 and optical logic 180, although it is appreciated that the console 110 can take one of a variety of forms and may include additional components (e.g., power supplies, ports, interfaces, etc.) that are not directed to aspects of the disclosure. An illustrative example of the console 110 is illustrated in U.S. Publication No. 2019/0237902, the entire contents of which are incorporated by reference herein. The processor 160, with access to the memory 165 (e.g., non-volatile memory or non-transitory, computer-readable medium), is included to control functionality of the console 110 during operation. As shown, the display 170 may be a liquid crystal diode (LCD) display integrated into the console 110 and employed as a user interface to display information to the clinician, especially during placement of the elongate medical device 120. In another embodiment, the display 170 may be separate from the console 110. Although not shown, a user interface is configured to provide user control of the console 110.

For both of these embodiments, the content depicted by the display 170 may constitute inter alia a two-dimensional (2D) or three-dimensional (3D) representation of the physical state (e.g., length, shape, form, and/or orientation) of the elongate medical device 120 computed from characteristics of reflected light signals 150 returned to the console 110. The reflected light signals 150 constitute light of a specific spectral width of broadband incident light 155 reflected back to the console 110. According to one embodiment of the disclosure, the reflected light signals 150 may pertain to various discrete portions (e.g., specific spectral widths) of broadband incident light 155 transmitted from and sourced by the optical logic 180, as described below.

Referring still to FIG. 1, the optical logic 180 is configured to support operability of the elongate medical device 120 and enable the return of information to the console 110, which may be used to determine the physical state associated with the elongate medical device 120, optionally including monitored electrical signals such as ECG signaling via an electrical signaling logic 181 that supports receipt and processing of the received electrical signals from the elongate medical device 120, or more specifically from the ECG electrode 125, (e.g., ports, analog-to-digital conversion logic, etc.). The physical state of the elongate medical device 120 may be based on changes in characteristics of the reflected light signals 150 received at the console 110 from the elongate medical device 120. The characteristics may include shifts in wavelength caused by strain on certain regions of the core fibers integrated within an optical fiber core 135 positioned within or operating as the elongate medical device 120, as shown below. As discussed herein, the optical fiber core 135 may be comprised of core fibers 1371-137M (M=1 for a single core, and M>2 for a multi-core), where the core fibers 1371-137M may collectively be referred to as core fiber(s) 137. Unless otherwise specified or the instant embodiment requires an alternative interpretation, embodiments discussed herein will refer to a multi-core optical fiber 135. From information associated with the reflected light signals 150, the console 110 may determine (through computation or extrapolation of the wavelength shifts) the physical state of the elongate medical device 120.

According to one embodiment of the disclosure, as shown in FIG. 1, the optical logic 180 may include a light source 182 and an optical receiver 184. The light source 182 is configured to transmit the incident light 155 (e.g., broadband) for propagation over the optical fiber(s) 147 included in the interconnect 145, which are optically connected to the multi-core optical fiber core 135 within the elongate medical device 120. In one embodiment, the light source 182 is a tunable swept laser, although other suitable light sources can also be employed in addition to a laser, including semi-coherent light sources, LED light sources, etc.

The optical receiver 184 is configured to (i) receive returned optical signals, namely reflected light signals 150 received from optical fiber-based reflective gratings (sensors) fabricated within each core fiber of the multi-core optical fiber 135 deployed within the elongate medical device 120 and (ii) translate the reflected light signals 150 into reflection data (from a data repository 192), namely data in the form of electrical signals representative of the reflected light signals including wavelength shifts caused by strain. The reflected light signals 150 associated with different spectral widths may include reflected light signals 151 provided from sensors (e.g., fiber optic Bragg gratings) positioned in the center core fiber (reference) of the multi-core optical fiber 135 and reflected light signals 152 provided from sensors positioned in the periphery core fibers of the multi-core optical fiber 135, as described below. Herein, the optical receiver 184 may be implemented as a photodetector, such as a positive-intrinsic-negative “PIN” photodiode, avalanche photodiode, or the like.

As shown, both the light source 182 and the optical receiver 184 are operably connected to the processor 160, which governs their operation. Also, the optical receiver 184 is operably coupled to provide the reflection data (from the data repository 192) to the memory 165 for storage and processing by reflection data classification logic 190. The reflection data classification logic 190 may be configured to (i) identify which core fibers pertain to which of the received reflection data (from the data repository 192) and (ii) segregate the reflection data stored within the data repository 192 provided from reflected light signals 150 pertaining to similar regions of the elongate medical device 120 or spectral widths into analysis groups. The reflection data for each analysis group is made available to shape sensing logic 194 for analytics.

According to one embodiment of the disclosure, the shape sensing logic 194 is configured to compare wavelength shifts measured by sensors deployed in each periphery core fiber at the same measurement region of the elongate medical device 120 (or same spectral width) to the wavelength shift at a center core fiber of the multi-core optical fiber 135 positioned along central axis and operating as a neutral axis of bending. From these analytics, the shape sensing logic 194 may determine the shape the core fibers have taken in three-dimensional space and may further determine the current physical state of the elongate medical device 120 in three-dimensional space for rendering on the display 170.

According to one embodiment of the disclosure, the shape sensing logic 194 may generate a rendering of the current physical state of the elongate medical device, based on heuristics or run-time analytics. For example, the shape sensing logic 194 may be configured in accordance with machine-learning techniques to access the data repository 192 with pre-stored data (e.g., images, etc.) pertaining to different regions of the elongate medical device 120 in which reflected light from core fibers have previously experienced similar or identical wavelength shifts. From the pre-stored data, the current physical state of the elongate medical device 120 may be rendered. Alternatively, as another example, the shape sensing logic 194 may be configured to determine, during run-time, changes in the physical state of each region of the multi-core optical fiber 135 based on at least (i) resultant wavelength shifts experienced by different core fibers within the optical fiber 135 and (ii) the relationship of these wavelength shifts generated by sensors positioned along different periphery core fibers at the same cross-sectional region of the multi-core optical fiber 135 to the wavelength shift generated by a sensor of the center core fiber at the same cross-sectional region. It is contemplated that other processes and procedures may be performed to utilize the wavelength shifts as measured by sensors along each of the core fibers within the multi-core optical fiber 135 to render appropriate changes in the physical state of the elongate medical device 120. Further exemplary details of the shape sensing system 102 may be taught by the U.S. Publication No. 2023-0081197 which is incorporated herein by reference in its entirety.

In some embodiments, the console 110 may further include electrical signaling logic 181, which is positioned to receive one or more electrical signals from the elongate medical device 120. The elongate medical device 120 may be configured to support both optical connectivity as well as electrical connectivity. The electrical signaling logic 181 receives the electrical signals (e.g., ECG signals) from the elongate medical device 120 via the conductive medium. The electrical signals may be processed by electrical signal logic 196, executed by the processor 160, to determine ECG waveforms for display, for example.

The MLS 101 enables the clinician to quickly locate and confirm the position and/or orientation of segments of the elongate medical device 120 during initial placement into and advancement through the vasculature of the patient. Specifically, the MILS 101 detects magnetic fields generated by a plurality of magnetic elements 127 coupled with the elongate medical device 120. In one embodiment, each of the magnetic elements 127 can be tracked using the teachings of one or more of the following U.S. Pat. Nos. 5,775,322; 5,879,297; 6,129,668; 6,216,028; and 6,263,230. The contents of the afore-mentioned U.S. patents are incorporated herein by reference in their entireties. The MLS 101 also detects an orientation of the magnetic elements 127, i.e., the direction in which each of the magnetic elements 127 is pointing, thus further assisting accurate placement of the elongate medical device 120. The MLS 101 further assists the clinician in determining when a malposition of a tip 123 of the elongate medical device 120 has occurred, such as in the case where the tip 123 has deviated from a desired venous path into another vein.

The magnetic elements 127 are disposed along the elongate medical device 120. In some embodiments, one or more magnetic elements 127 may be disposed at the distal tip 123 of the elongate medical device 120 and additional magnetic elements 127 may be dispersed along a length of the elongate medical device 120 extending away from the distal tip 123. The elongate medical device 120 may include 1, 2, 3, 4, 5, or more magnetic elements 127. The elongate medical device 120 may also include an ECG electrode 125 at the distal tip 123.

Each magnet element 127 may be longitudinal in shape and each magnet element 127 may disposed parallel with (or tangent to) the elongate medical device 120. In other words, each magnetic element 127 may be attached to the elongate medical device 120 so that a longitudinal axis of the magnetic element 127 is aligned with a longitudinal axis of the elongate medical device 120. Each magnet element 127 includes a north magnetic pole 128A and a south magnetic pole 128B located at opposite ends of the magnetic element 127. In some embodiments, each north magnetic pole 128A may point in a proximal direction and each south magnetic pole 128A may point in a distal direction or vice versa. In some embodiments, the magnetic elements 127 may be permanent magnets. In further embodiments, the magnetic elements 127 may include magnetized portions of a ferrous material. In other embodiments, the magnetic elements 127 may vary from the design in not only shape, but also composition, number, size, magnetic type, and position along the elongate medical device 120.

For example, in one embodiment, the plurality of magnetic elements 127 may be replaced with an electromagnetic assembly, such as an electromagnetic coil, which produces a magnetic field for detection by the magnetic field sensor 129. Another example of an assembly usable here can be found in U.S. Pat. No. 5,099,845 entitled “Medical Instrument Location Means,” which is incorporated herein by reference in its entirety. Yet other examples of stylets including magnetic elements that can be employed with the MLS 101 can be found in U.S. application Ser. No. 11/466,602, filed Aug. 23, 2006, and entitled “Stylet Apparatuses and Methods of Manufacture,” published as U.S. Publication No. 2007/0049846, which is incorporated herein by reference in its entirety. These and other variations are therefore contemplated by embodiments of the disclosure.

The magnetic field sensor 129 is employed by the MLS 101 during operation to detect magnetic fields defined by the magnetic elements 127 of the elongate medical device 120. The MLS 101 includes a magnetic field sensor 129 configured to sense a magnetic field defined by each magnetic element 127 which sensing includes determining a position of the magnetic element 127 in three-dimensional space with respect to the magnetic field sensor 129. The sensing may further include determining an orientation of the magnetic element 127, i.e., the direction of the north and south poles 128A, 128B. An exemplary system for coupling magnetic elements with a medical device, inserting the medical device within a human body, and determining the position and orientation the magnetic elements with the human body is shown and described in U.S. Pat. No. 8,388,541 entitled “Integrated System for Intravascular Placement of a Catheter,” the entire contents of which is incorporated by reference herein. As the magnetic elements 127 are coupled with the elongate medical device 120, the positions of the individual magnetic elements 127 define a position of the elongate medical device 120.

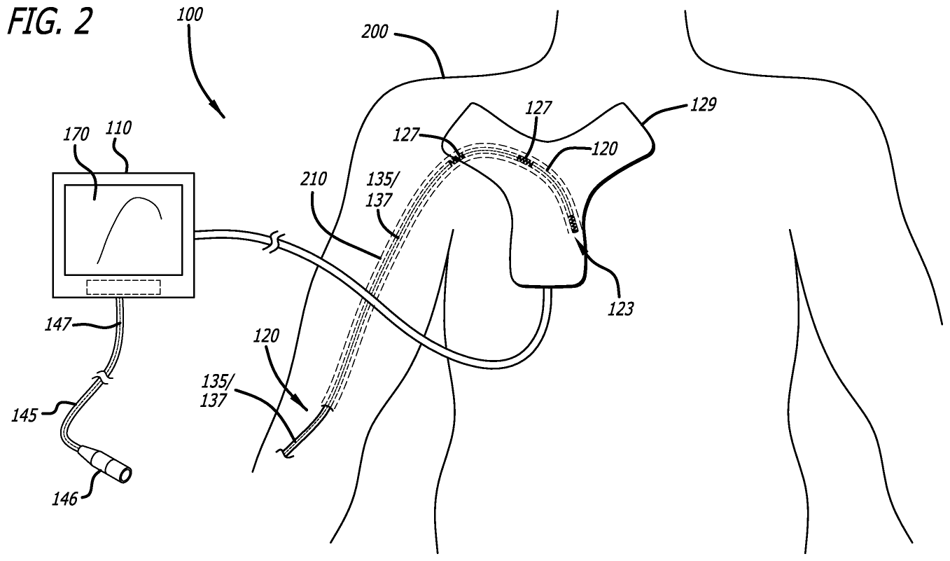

According to one embodiment of the disclosure, the magnet sensing logic 197 may generate a rendering of the positions and orientations of the magnetic elements 127 or the position of the elongate medical device 120 as a whole. For example, a magnetic element 127 may define a magnetic field which is sensed by the magnetic field sensor 129. The magnetic sensing logic 197 receives magnetic field data from the magnetic field sensor 129 and therefrom determines a three-dimensional position of the magnetic element 127. As the magnetic field is defined by north and south poles 128A, 128B, magnetic sensing logic 197 also determines the orientation of the magnetic element 127. As each magnetic element 127 is coupled with an attachment segment of the elongate medical device 120, the magnet sensing logic 197 determines a three-dimensional position of the attachment segment of the elongate medical device 120. Further, as each longitudinal axis of the magnetic element 127 is alignment with a longitudinal axis of the attachment segment of the elongate medical device 120, the magnet sensing logic 197 determines a three-dimensional direction/orientation of the attachment segment. By way of summary, the shape sensing logic 194 determines the three-dimensional shape of the elongate medical device 120 and the magnet sensing logic 197 determines the three-dimensional position and orientation of the elongate medical device 120. Hence, the shape sensing logic 194 in combination with the magnet sensing logic 197, determines the three-dimensional shape, position, and orientation of the elongate medical device 120. The memory 165 further includes tracking record logic 198 that is generally configured to store tracking records, i.e., a tracking record for each placement event of the elongate medical device 120 within an anatomical pathway 210 (see FIG. 2). The memory 165 further includes anatomical pathway logic 199 as further described below.

FIG. 2 illustrates the medical device tracking system 100 during operation with a patient 200. The elongate medical device 120 is shown advanced along an anatomical pathway 210 (e.g., a vasculature) within the patient 200. The magnetic field sensor 129 is placed on the chest of the patient 200. In some embodiments, the magnetic field sensor 129 may be located at a defined location with respect to specific anatomical land marks, such as the sternum, or the collar bone, for example. As such, as the magnetic elements 127 are coupled with the elongate medical device 120, detection by the magnetic field sensor 129 of the magnetic fields defined by the magnetic elements 127 determines the position and/or orientation of the elongate medical device 120 within the patient 200. As one of the magnetic elements 127 may be co-terminal with the distal tip 123 of the elongate medical device 120, detection by the magnetic field sensor 129 of the magnetic field defined by the magnetic element 127 located at the distal tip 123 may determine the position and/or orientation of the distal tip 123.

In greater detail, the magnetic field sensor 129 is operably connected to the console 110 of the system 100. The console 110 includes a magnetic signal module 185 including connection ports 186, as shown in FIG. 1. The magnetic signal module 185 receives magnetic signals via the ports 186 from the magnetic field sensor 129 and converts the magnetic signals into magnetic data for processing by the magnetic sensing logic 197. In some embodiments, the magnetic signal module 185 may be located separate from the console 110. In such embodiments, the magnetic signal module 185 may include a transmitter for delivering magnetic data to the console 110.

Detection by the magnetic field sensor 129 of the magnetic elements 127 is graphically displayed on the display 170 of the console 110 during operation. In this way, a clinician placing the elongate medical device 120 is able to generally determine the location of the elongate medical device 120 within the patient vasculature relative to the magnetic field sensor 129 and detect when malposition of the elongate medical device 120, such as advancement along an undesired vein, is occurring.

During advancement along a patient vasculature, the elongate medical device 120 receives broadband incident light 155 from the console 110 via optical fiber(s) 147 within the interconnect 145, where the incident light 155 propagates along the core fibers 137 of the multi-core optical fiber 135 of the elongate medical device 120. According to one embodiment of the disclosure, the connector 146 of the interconnect 145 terminating the optical fiber(s) 147 may be coupled to the optical-based connector 144, which may be configured to terminate the core fibers 137 deployed within the elongate medical device 120. Such coupling optically connects the core fibers 137 of the elongate medical device 120 with the optical fiber(s) 147 within the interconnect 145. The optical connectivity is needed to propagate the incident light 155 to the core fibers 137 and return the reflected light signals 150 to the optical logic 180 within the console 110 over the interconnect 145. As described below in detail, the physical state of the elongate medical device 120 may be ascertained based on analytics of the wavelength shifts of the reflected light signals 150.

Further during advancement along the anatomical pathway 210, as the magnetic elements 127 are attached and aligned with the elongate medical device 120, the magnetic field sensor 129 determines the three-dimensional position and orientation of the elongate medical device 120 as a whole, or more specifically the position and/or orientation of each of the magnetic elements 127 coupled with the elongate medical device 120, with respect to the magnetic field sensor 129. As the magnetic field sensor 129 is applied to the patient 200 at a defined location on the patient 200 (e.g., at a location with respect to patient's heart), the three-dimensional position and orientation of the elongate medical device 120 within the patient 200 may be ascertained by sensing the magnetic fields defined by the magnetic elements 127. By way of summary, the shape sensing system 102 of the system 100 defines the shape of the elongate medical device 120, and the MLS 101 determines the location and/or orientation of the elongate medical device 120 within the patient 200 with respect to the magnet field sensor 129. The system 100 may be configured to track the position of the elongate medical device 120 with respect to a predicted anatomical pathway stored in the memory 165 as further described below.

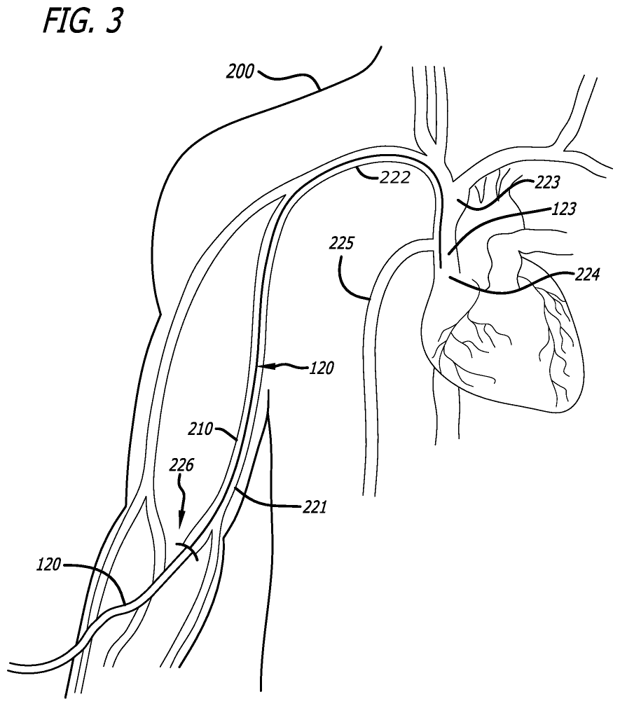

FIG. 3 illustrates the elongate medical device 120 disposed along an exemplary anatomical pathway 210 that includes a vasculature of the patient 200. In the illustrated embodiment, the anatomical pathway 210 includes vasculature consistent with placement of the peripherally inserted central catheter (PICC). As such, by way of one example, the anatomical pathway 210 may include an insertion site 226, a brachial vein 221, a subclavian vein 222, and the superior vena cava 223. In some embodiments, the anatomical pathway 210 may include a lower one-third (⅓) of the Superior Vena Cava 223, the Cavo-atrial junction 224, or the azygos vein 225. Anatomical pathways other than vascular pathways are also considered, such as a urinary tract, or a drainage tube pathway, for example.

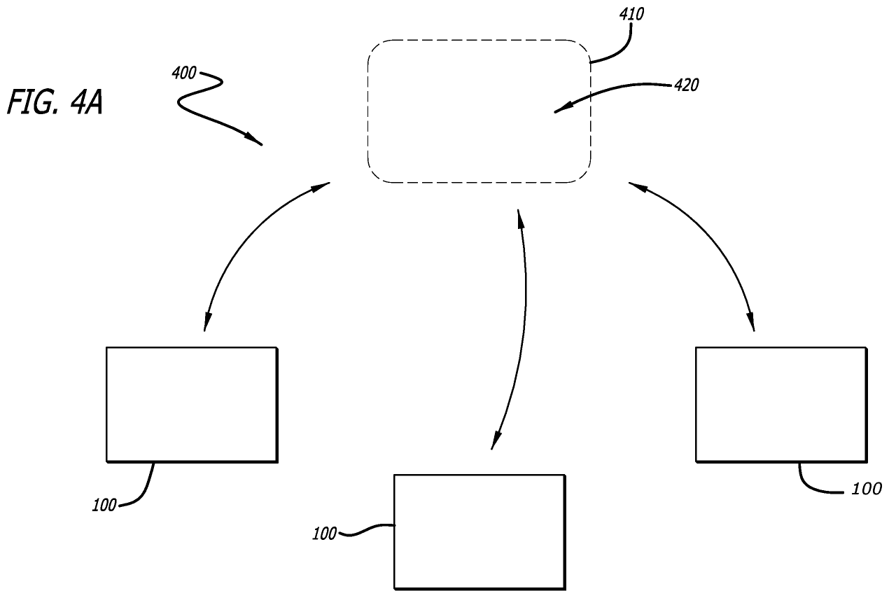

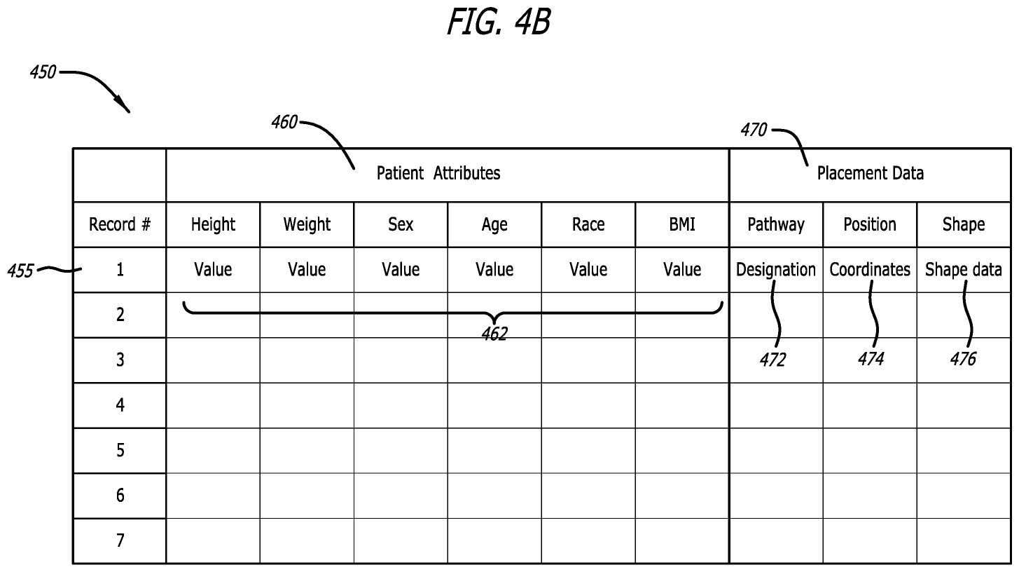

FIG. 4A illustrates a medical network system 400, according to some embodiments. The medical network system 400 includes a plurality of medical device tracking systems 100 coupled with (e.g., wirelessly coupled with) a system module 410 executing a logic module 420. The system module 410 may include any type of a computing system, such as a network server, for example. As such, the system module 410 may include a console (not shown) including a processor and a memory (e.g., a non-transitory computer-readable medium) having the logic module 420 stored thereon. In other embodiments, the system module 410 may include cloud based internet application. Each of the medical device tracking systems 100 may exchange data (i.e., transmit and receive) with the system module 410.

The logic module 420 performs operations of the network system 400. In general terms, the logic module 420 receives tracking records from the medical device tracking systems 100 and processes the tracking records (i.e., data of the tracking records) in accordance with machine learning and/or artificial intelligence techniques as further described below. In some embodiments, the logic module 420 may include a database that includes tracking records linked with patient attributes.

FIG. 4B illustrates an exemplary database table (table) 450 as may be stored in the memory of the system module 410. As shown, each tracking record 455 may include patient attributes 460 for a specific patient and placement data 470 for the elongate medical device 120 with the specific patient 200. The patient attributes 460 may include any suitable attributes of the patient, such as height, weight, sex, age, race, body mass index (BMI), or any combination thereof. Other patient attributes may also be considered. More specifically, the table 450 may include a value (or entry) for each of the patient attributes 460, where the values for each of the patient attributes 460 define a patient value set 462 for the specific patient.

The placement data 470 may include the position of the elongate medical device 120 after the elongate medical device 120 is properly placed within the anatomical pathway 210. The placement data 470 includes a designation (or name) for the anatomical pathway 210, such as a “PICC pathway”, for example. The placement data 470 for the position of the elongate medical device 120 may include any suitable data that sufficiently defines the position of the elongate medical device 120 within the patient 200. In some embodiments, the position of the elongate medical device 120 may include 2-dimensional or three-dimensional coordinates 474 for each magnetic element 127 with respect to the magnetic field sensor 129 where the magnetic field sensor 129 is placed on the patient at a defined location with respect to specific landmarks of the patient 200 as described above.

In some embodiments, the position of the elongate medical device 120 may also include an orientation of each magnetic element 127. In such embodiments, the position may include a 2-dimensional or three-dimensional vector for all or any subset of the magnetic elements 127. In accordance with such embodiments, a vector for a magnetic element 127 located adjacent the distal tip 123 of the elongate medical device 120 may indicate a direction of the distal tip 123. In some embodiments, the placement data 470 may also include the shape 476 of the elongate medical device 120, i.e., the shape of the optical fiber 135 extending along the elongate medical device 120. The shape 476 may include shape data consistent with sensing the shape of the optical fiber 135, such as specific spectral widths of broadband incident light reflected back by fiber optic Bragg gratings disposed along the optical fiber 135, for example.

The logic module 420 may process the tracking records in accordance with machine learning and/or artificial intelligence techniques to define a correlation (or a correlation algorithm) between the patient attributes 460 and the placement data 470. In some embodiments, the techniques may include statistical and/or linear regression techniques. Having defined the correlation, the logic module 420 may be configured to predict (e.g., map) an anatomical pathway based on the correlation and a patient value set 462 of the patient attributes 460. In use, the logic module 420 may (i) receive a new patient value set of the patient attributes 460 from a medical device tracking system 100, (ii) process the new patient value set using correlation algorithm to define a predicted anatomical pathway, and (iii) transmit the predicted anatomical pathway back to the medical device tracking system 100.

In some embodiments, processing the tracking records to define the correlation may yield a confidence level for the correlation and/or the predicted anatomical pathway. The confidence level may be related to the quantity of tracking records processed to define the correlation. As may be appreciated, a greater quantity of tracking records may yield a higher confidence level. As such, the logic module 420 may subsequently receive additional tracking records, and the logic module 420 may process the additional tracking records to yield a higher confidence level for the correlation. In some embodiments, the logic module 420 may continuously process tracking records as they are received to continuously update the correlation and/or confidence level.

In some embodiments, the confidence level for the anatomical pathway may multiple confidence levels different segments of the anatomical pathway. For example, a confidence level adjacent the Superior Vena Cava 223 may be different than a confidence level along the brachial vein 221. In some embodiments, the logic module 420 may transmit the confidence level back to the medical device tracking system 100 along with the predicted anatomical pathway.

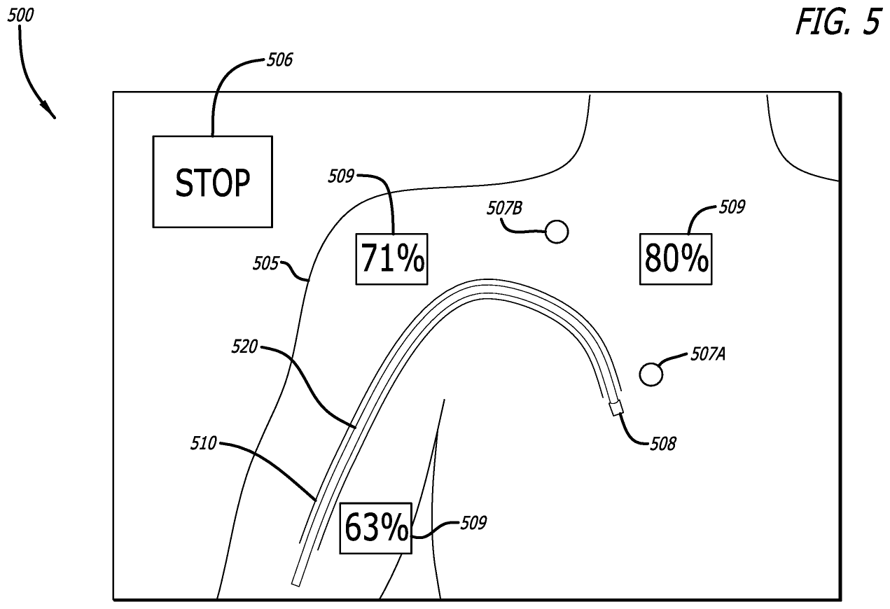

FIG. 5 illustrates an exemplary screen shot 500 as may be depicted on the display 170, according to some embodiments. The anatomical pathway logic 199 of the system 100 (see FIG. 1) is generally configured to govern the data exchange between the medical device tracking system 100 and the system module 410. The anatomical pathway logic 199 may transmit the tracking records to the system module 410 for processing by the logic module 420 as described above.

In the illustrated embodiment, the anatomical pathway logic 199 may transmit the new patient value set for the patient attributes 460 to the system module 410 so that the logic module 420 may define a predicted anatomical pathway for the new patient value set as described above. The logic module 420 transmits the predicted anatomical pathway to the medical device system 100 so that the anatomical pathway logic 199 may depict the predicted anatomical pathway image 520 (i.e., an image of the predicted anatomical pathway) on the display 170. In some embodiments, the anatomical pathway logic 199 may depict an exemplary patient image 505 on the display 170 together with the predicted anatomical pathway image 520, so that the user may view the predicted anatomical pathway image 520 in relation to the exemplary patient image 505. In some embodiments, the anatomical pathway logic 199 may also depict a target 508 in relation to the exemplary patient image 505. In some embodiments, the target 508 may represent a desired placement location for the distal tip 123.

The anatomical pathway logic 199 may track the position of the elongate medical device 120 in real time during advancement of the elongate medical device 120 with respect to the anatomical pathway image 520. Accordingly, the anatomical pathway logic 199 may depict an elongate medical device image 520 in relation to the anatomical pathway image 520. As such, during advancement of the elongate medical device 120, the user may determine when the elongate medical device image 520 follows the predicted anatomical pathway image 510 and when the elongate medical device image 120 deviates from the anatomical pathway image 510. The anatomical pathway logic 199 may also indicate a number (e.g., 1, 2, 3, or more) confidence levels 509 on the display 170 in relation to segments or portions the anatomical pathway image 520. In some embodiments, anatomical pathway logic 199 may depict a number of anatomical land marks on the display 170, such as the heart 507A or a jugular vein 507B, for example, in relation to exemplary patient image 505.

In some embodiments, the anatomical pathway logic 199 may automatically detect when the elongate medical device 120 deviates from the predicted anatomical pathway. In such embodiments, the anatomical pathway logic 199 may provide a notification to the user. The notification may take various forms. For example, as illustrated, the notification may include a visual notification 506 on the display 170. The visual notification 506 may include a text message including an instruction, such as an instruction to stop advancement when the deviation from the predicted anatomical pathway is detected, for example. In some embodiments, the text message may include a position or a displacement direction of the elongate medical device 120 when the elongate medical device 120 deviates from the predicted anatomical pathway. In some embodiments, the visual notification 506 may include a color change, such as a change from green to yellow to red when the elongate medical device 120 deviates from the predicted anatomical pathway, for example. Other types of color change are also considered, such as a heat map, for example. The notification may also or alternatively include an audible notification, such as an alarm when the elongate medical device 120 deviates from the predicted anatomical pathway. In some embodiments, the anatomical pathway logic 199 may specifically detect a deviation from a predicted anatomical pathway that includes the azygos vein 225. In some embodiments, the anatomical pathway logic 199 may specifically detect a deviation that includes a jugular or contralateral deviation.

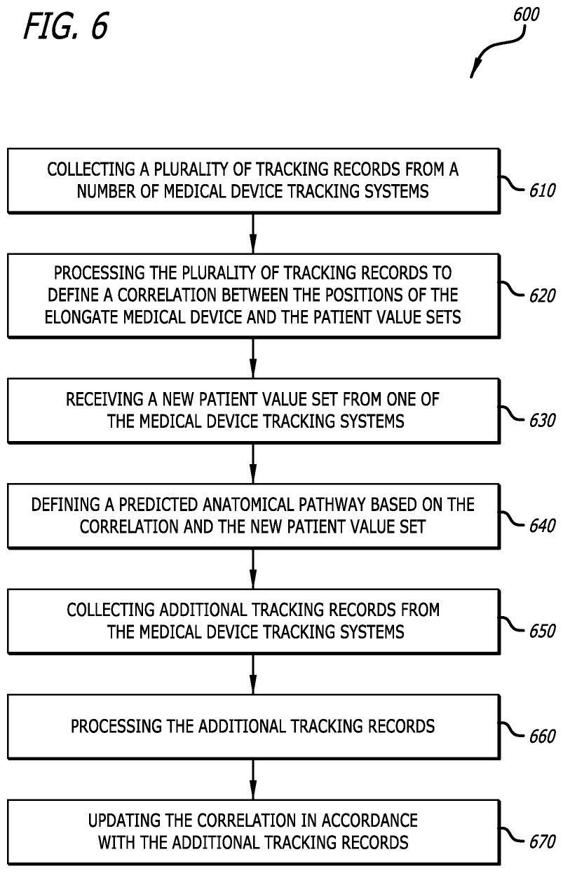

FIG. 6 illustrates a block diagram of the system method 600 of mapping an anatomical pathway for an elongate medical device that may, according to some embodiments, include all or any subset of the following steps, actions, or processes. The system method 600 may include collecting a plurality of tracking records from a number of medical device tracking systems (block 610). Each tracking record includes (i) a patient value set having a value for each of a number of physical attributes of the patient and (ii) a position of an elongate medical device disposed along a defined anatomical pathway within the patient. The position of the elongate medical device is linked with the patient value set.

In some embodiments of the system method 600, the medical device tracking system includes a magnetic tracking system, including a number of magnetic elements disposed along the elongate medical device, where the magnetic elements define a number of magnetic fields; and a number of magnetometers placed on the patient, where the magnetometers are configured to detect the magnetic fields. In such embodiments, detecting the magnetic fields determines the positions of the magnetic elements in three-dimensional space with respect to the magnetometers, thereby determining the position of the elongate medical device within the patient.

In some embodiments of the system method 600, the medical device tracking system includes a fiber optic shape sensing system that includes an optical fiber extending along the elongate medical device. The optical fiber includes a number of optical fiber cores, where each optical fiber core includes a plurality of fiber Bragg gratings configured to determine a strain of the optical fiber, and further where the strain of the optical fiber determines a three-dimensional shape of the elongate medical device within the patient. In such embodiments, the tracking record includes the shape of the elongate medical device, and processing the plurality of tracking records to define a correlation between the positions of the elongate medical device and the patient value sets includes defining a correlation between the shapes of the elongate medical device and the patient value sets.

The system method 600 may further include processing the plurality of tracking records to define a correlation between the positions of the elongate medical device and the patient value sets (block 620). In some embodiments, defining the correlation may utilize machine learning and/or artificial intelligence techniques.

The system method 600 may further include receiving a new patient value set from one of the medical device tracking systems (block 630) and defining a predicted anatomical pathway based on the correlation and the new patient value set (block 640).

In some embodiments, the system method 600 may further include (i) collecting additional tracking records from the medical device tracking systems (block 650), (ii) processing the additional tracking records (block 660), and (iii) updating the correlation in accordance with the additional tracking records (block 670). In some embodiments, updating the correlation includes updating a confidence level for the predicted anatomical pathway. In some embodiments, the system method 600 may include continuously processing the additional tracking records as they are received and continuously updating the confidence level.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A medical system, comprising:
an elongate medical device configured for advancement along an anatomical pathway within a patient;
a tracking system configured to track the elongate medical device within the patient, the tracking system comprising a console including a processor and a memory having logic stored thereon that, when executed by the processor performs operations that include tracking a position the elongate medical device with respect to a predicted anatomical pathway stored in the memory.

2. The system according to claim 1, wherein the anatomical pathway includes a vascular pathway.

3. The system according to claim 2, wherein the vascular pathway includes at least one of a lower one third of a superior vena cava, or a Cavo-atrial junction.

4. The system according to claim 2, wherein the vascular pathway includes an azygos vein.

5. The system according to claim 1, wherein the predicted anatomical pathway is defined based on a number of patient attributes.

6. The system according to claim 1, wherein the operations further include providing a notification when the position the elongate medical device deviates from the predicted anatomical pathway.

7. The system according to claim 5, wherein the operations further include:
transmitting the patient attributes to a network module, and
receiving the predicted anatomical pathway from the network module.

8. The system according to claim 7, wherein the operations further include:
making a record of the position of the elongate medical device after advancement along the anatomical pathway; and
transmitting the record to the network module.

9. The system according to claim 1, wherein the tracking system includes a magnetic tracking system, comprising:
a number of magnetic elements disposed along the elongate medical device, the magnetic elements defining a number of magnetic fields; and
a number of magnetometers placed on the patient and coupled with console, the magnetometers configured to detect the magnetic fields,
wherein tracking the position the elongate medical device includes determining positions of the magnetic elements in three-dimensional space with respect to the magnetometers.

10. The system according to claim 9, wherein the tracking system further includes a fiber optic shape sensing system comprising an optical fiber extending along the elongate medical device, wherein:
the optical fiber is operatively coupled with the console,
the optical fiber includes a number of optical fiber cores, each optical fiber core including a plurality of fiber optic Bragg gratings configured to determine a strain in the optical fiber, and
tracking the position the elongate medical device further includes determining a three-dimensional shape of the elongate medical device based on the strain.

11. A medical network system, comprising:
a number of medical device tracking systems communicatively coupled with a network system module that comprises a console including a processor and a memory having logic stored thereon that, when executed by the processor performs operations, including:
receiving a plurality of tracking records from the medical device tracking systems, each tracking record including:
a patient value set having a value for each of a number of physical attributes of a patient; and
a position of an elongate medical device disposed along a defined anatomical pathway within the patient, the position of the elongate medical device linked with the patient value set; and
processing the plurality of tracking records to define a correlation between the positions of the elongate medical device and the patient value sets.

12. The system according to claim 11, wherein each patient value set includes a number of physical of attributes of the patients, the physical of attributes including height, weight, sex, age, body mass index, race, or any combination thereof.

13. The system according to claim 12, wherein the operations further include:
receiving a new patient value set from one of the medical device tracking systems; and
transmitting a predicted anatomical pathway to the one of the medical device tracking systems, the predicted anatomical pathway based on:
the correlation between the positions of the elongate medical device and the patient value sets, and
the new patient value set.

14. The system according to claim 13, wherein the predicted anatomical pathway includes a confidence level, the operations further including:
receiving additional tracking records from the medical device tracking systems; and
processing the additional tracking records to further define the correlation between the positions of the elongate medical device and the patient value sets, thereby increasing the confidence level.

15. The system according to claim 11, wherein each of the medical device tracking systems includes a magnetic tracking system, comprising:
a number of magnetic elements disposed along the elongate medical device, the magnetic elements defining a number of magnetic fields; and
a number of magnetometers placed on the patient and coupled with console, the magnetometers configured to detect the magnetic fields,
wherein detecting the magnetic fields determines the positions of the magnetic elements in three dimensional space with respect to the magnetometers, thereby determining the position of the elongate medical device within the patient.

16. The system according to claim 15, wherein each of the medical device tracking systems includes a fiber optic shape sensing system, comprising an optical fiber extending along the elongate medical device, wherein:
the optical fiber is operatively coupled with the console,
the optical fiber includes a number of optical fiber cores,
each optical fiber core includes a plurality of fiber Bragg gratings configured to determine a strain of the optical fiber,
the strain of the optical fiber determines a three-dimensional shape of the elongate medical device within the patient,
the tracking record includes the shape of the elongate medical device, and
processing the plurality of tracking records to define a correlation between the positions of the elongate medical device and the patient value sets includes defining a correlation between the shapes of the elongate medical device and the patient value sets.

17. A system method of mapping an anatomical pathway for an elongate medical device, comprising:
collecting a plurality of tracking records from a number of medical device tracking systems, each tracking record including:
a patient value set having a value for each of a number of physical attributes of a patient; and
a position of an elongate medical device disposed along a defined anatomical pathway within the patient, the position of the elongate medical device linked with the patient value set; and
processing the plurality of tracking records to define a correlation between the positions of the elongate medical device and the patient value sets;
receiving a new patient value set from one of the medical device tracking systems; and
defining a predicted anatomical pathway based on the correlation and the new patient value set.

18. The system method according to claim 17, further comprising:
collecting additional tracking records from the medical device tracking systems;
processing the additional tracking records; and
updating the correlation in accordance with the additional tracking records.

19. The system method according to claim 17, wherein the medical device tracking systems include a magnetic tracking system, comprising:
a number of magnetic elements disposed along the elongate medical device, the magnetic elements defining a number of magnetic fields; and
a number of magnetometers placed on the patient, the magnetometers configured to detect the magnetic fields,
wherein detecting the magnetic fields determines the positions of the magnetic elements in three-dimensional space with respect to the magnetometers, thereby determining the position of the elongate medical device within the patient.

20. The system method according to claim 19, wherein the medical device tracking system includes a fiber optic shape sensing system comprising an optical fiber extending along the elongate medical device, wherein:
the optical fiber includes a number of optical fiber cores,
each optical fiber core includes a plurality of fiber Bragg gratings configured to determine a strain of the optical fiber,
the strain of the optical fiber determines a three-dimensional shape of the elongate medical device within the patient,
the tracking record includes the shape of the elongate medical device, and
processing the plurality of tracking records to define a correlation between the positions of the elongate medical device and the patient value sets includes defining a correlation between the shapes of the elongate medical device and the patient value sets.
